(19) United States
(12) Patent Application Publication
(10) Pub. No.: US 2025/0359732 A1
(43) Pub. Date: Nov. 27, 2025

(54) MEDICAL DEVICE SHAFTS, COMPONENTS, ASSEMBLIES, AND RELATED METHODS OF MANUFACTURING AND/OR ASSEMBLING TO PREVENT LEAKS

(71) Applicant: Boston Scientific Scimed, Inc., Maple Grove, MN (US)

(72) Inventors: Lisa M. SANGREE, Boston, MA (US); Evan WILDER, Waltham, MA (US); Thomas BIANCHI, Palmer, MA (US); Daragh NOLAN, Via Youghal, Co. Waterford (IE); Daniel J. GRASSO, Franklin, MA (US)

(73) Assignee: Boston Scientific Scimed, Inc., Maple Grove, MN (US)

(21) Appl. No.: 19/214,461

(22) Filed: May 21, 2025

Related U.S. Application Data
(60) Provisional application No. 63/651,500, filed on May 24, 2024.

(51) Int. Cl. A61B 1/00; A61B 1/012; A61B 90/00

(52) U.S. Cl. CPC A61B 1/00128; A61B 1/00066; A61B 1/012; A61B 90/03; A61B 2090/034

(57) ABSTRACT

A medical device includes a handle including a port that includes a port lumen. The medical device includes a shaft extending from a portion of the handle. A first portion of the shaft includes a plurality of first lumens and a second lumen, and a second portion of the shaft includes only the second lumen. The second portion of the shaft is proximal to the first portion of the shaft. The medical device also includes a coupling assembly that couples a proximal end of the second portion of the shaft to the port. The coupling assembly forms a seal between the proximal end of the shaft and the port to fluidly connect the port lumen to the second lumen.

### CROSS-REFERENCE TO RELATED APPLICATIONS

This application claims the benefit of priority to U.S. Provisional Application No. 63/651,500, filed on May 24, 2024, which is incorporated by reference herein in its entirety.

### TECHNICAL FIELD

This disclosure generally relates to medical device shafts, components, assemblies, and related methods of manufacturing and/or assembling to prevent leaks. In some aspects, this disclosure relates to seals and/or internal components of a handle that couple a working channel of a shaft to a port (e.g., a biopsy port) on an exterior of the handle, and related methods of manufacturing and/or assembling.

### BACKGROUND

Medical devices are often inserted into the body to perform a therapeutic and/or diagnostic procedure inside a subject's body. An example of such a device is an endoscope or other type of scope or insertion device, which includes a handle and an insertion portion extending distally from the handle. The insertion portion of an endoscope may be inserted into the subject's body through an opening (e.g., a natural opening or an incision) and is delivered to a site inside the body, for example, through a body lumen. The insertion portion of an endoscope or other insertion device includes a shaft that extends from outside the body to the treatment site.

The shaft includes a working channel or internal lumen. The shaft often also includes a number of other channels or lumens, for example, for delivering irrigation, for applying suction, for receiving pull or deflection wires, electrical connections, or other components, etc. The shaft is coupled to a handle, which may control various aspects of the shaft, including movement of the pull or deflection wires, the delivery of irrigation, the application of suction, the activation/deactivation of the electrical connections, etc. The working channel or internal lumen of the shaft is often coupled (e.g., fluidly coupled) to a port on the handle, such as a biopsy port on an exterior of the handle.

### SUMMARY

Examples of this disclosure relate to, among other things, medical devices having a handle and a shaft or insertion portion. For example, examples of this disclosure relate to devices, components, assemblies, and methods of helping to avoid and/or prevent a leak when coupling a portion of the insertion portion or shaft (e.g., a working channel or lumen) to a portion of the handle (e.g., to a biopsy port).

In an example, a medical device may include a handle including a port that includes a port lumen. The medical device may include a shaft extending from a portion of the handle. A first portion of the shaft may include a plurality of first lumens and a second lumen, and a second portion of the shaft may include only the second lumen. The second portion of the shaft may be proximal to the first portion of the shaft. The medical device may also include a coupling assembly that couples a proximal end of the second portion of the shaft to the port. The coupling assembly may form a seal between the proximal end of the shaft and the port to fluidly connect the port lumen to the second lumen.

Any examples described herein may have any of these features alone or in combination. The coupling assembly may include a ring and a connector. The ring may include a proximal surface coupled to a distal portion of the port. The connector may be configured to form a snap fit coupling to the ring to couple the proximal end of the second portion of the shaft to the port. The ring may include a distal ledge, a proximal ledge, and a plurality of arms. Each of the plurality of arms may extend proximally from the proximal ledge to the proximal surface of the ring. The ring may include a projection that extends distally from the proximal surface of the ring radially within at least a portion of the distal ledge and the proximal ledge. The adjacent arms of the plurality of arms may be disposed at an interval defining a plurality of openings between the proximal ledge and the proximal surface. The connector may include a connector ring, a through-hole, and a plurality of fingers. The plurality of fingers may extend proximally from the connector ring, and the adjacent fingers may be spaced apart by an interval. Each finger of the plurality of fingers may include a raised ledge and a stop surface in a proximal portion of the respective finger.

The coupling assembly may include at least one internal ring, an outer clamp, and a hypotube. The at least one internal ring may include an opening positioned on a radially outer surface of a portion of the second portion of the shaft. The outer clamp may be configured to compress the at least one internal ring to couple the second portion of the shaft to the port, and a proximal end of the at least one internal ring may abut a distal end of the port. The at least one internal ring may include a plurality of internal rings, and the plurality of internal rings may be positioned on a radially outer surface of a portion of the second portion of the shaft. The outer clamp may compress the plurality of internal rings, and one of the plurality of internal rings may be a proximal internal ring abutting a distal end of the port. The coupling assembly may include a hypotube, and a proximal portion of the hypotube may be positioned within a distal portion of the port. A distal portion of the hypotube may be positioned within a proximal portion of the second lumen of the shaft. The coupling assembly may further include an adhesive, and the adhesive may be positioned at least between a portion of an outer surface of the hypotube and a portion of an inner surface of the port lumen. The port lumen may include a distal portion and a proximal portion. At least a portion of the proximal portion of the port lumen may be narrower than at least a portion of the distal portion of the port lumen to form one or more abutments. A proximal portion of the second portion of the shaft may extend through a portion of the port lumen, and a proximal end face of the second portion of the shaft may abut one of the one or more abutments. The port may include a suction port extending from the port lumen, and a proximal end of the hypotube may be distal to the suction port.

In another example, a medical device may comprise a handle. The handle may include a port, and the port may include a port lumen. The medical device may include a coupling assembly including a ring and a connector. The ring may include a distal ledge, a proximal ledge, a plurality of arms, and a proximal surface, and the proximal surface of the ring may be coupled to a distal portion of the port. The connector may include a connector ring, and a plurality of fingers may each include a raised ledge and a plurality of stop surfaces. The connector may be coupled to the ring. The plurality of fingers may be positioned radially inward along the proximal ledge such that the proximal ledge may reach stop surfaces of each of the plurality of fingers. Furthermore, the raised ledge may engage with a surface of the proximal ledge of the ring to form the coupling assembly.

Any of the devices disclosed herein may include any of the following features in any combination. The ring may include a projection that extends from the proximal surface of the ring radially within at least a portion of the distal ledge and the proximal ledge. The connector ring may include an opening that at least partially overlaps with the projection when the connector is coupled to the ring. The projection may taper from a proximal end to a distal end. The projection may include an opening extending through a length of the projection, and the opening of the projection is fluidly connected to the port lumen. The medical device may also include a shaft. The shaft may extend from a portion of the handle, and a first portion of the shaft includes a plurality of first lumens and a second lumen. The second portion of the shaft only includes the second lumen. A proximal portion of the second portion the shaft may be positioned around the distal end of the projection, and the coupling assembly may form a seal between the proximal end of the shaft and the port.

In another example, the medical device may include a handle. The handle may include a port, and the port may include a port lumen. The shaft may extend from a portion of the handle, and a first portion of the shaft may include a plurality of first lumens and a second lumen. A second portion of the shaft may only include the second lumen. The medical device may also include a hypotube. A proximal portion of the hypotube may be positioned within a distal portion of the port, and a distal portion of the hypotube may be positioned within a proximal portion of the second lumen of the shaft. A coupling assembly may include at least one outer crimping tube and an outer tube. The outer crimping tube may be positioned on the outer tube, and the outer crimping tube and the outer tube may form a seal between the second portion of the shaft and the port to fluidly connect the second lumen to the port lumen.

### BRIEF DESCRIPTION OF THE FIGURES

The accompanying drawings, which are incorporated in and constitute a part of this specification, illustrate exemplary aspects of the disclosure and together with the description, explain the principles of the disclosure.

FIG. 1A is a perspective view of a proximal portion of a medical device, according to aspects of this disclosure.

FIG. 1B is a perspective view of a distal portion of the medical device, according to aspects of this disclosure.

FIG. 2A illustrates an interior portion of a handle of the medical device.

FIG. 2B is a cross-sectional view of a portion of an insertion portion or shaft of the medical device, according to aspects of this disclosure.

FIG. 3A illustrates a proximal portion of the insertion portion or shaft of the medical device coupled to a port on the handle including a coupling assembly, according to aspects of this disclosure.

FIG. 3B illustrates a perspective view of the coupling assembly of FIG. 3A.

FIG. 4 illustrates a perspective view of a coupling system for use in the proximal portion of the handle of the medical device, according to aspects of this disclosure.

FIG. 5A illustrates a perspective view of another coupling assembly for use in a portion of the handle of the medical device, with the coupling assembly in a partially exploded configuration.

FIG. 5B illustrates a perspective view of the coupling assembly of FIG. 5A in an assembled configuration.

FIG. 5C illustrates a perspective view of a component of coupling assembly of FIG. 5A and FIG. 5B.

FIGS. 6A and 6B illustrate a perspective views of another coupling assembly for use in a portion of the handle of the medical device.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a subject (e.g., patient). By contrast, the term “proximal” refers to a portion closest to the user when introducing the device into the subject.

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. Additionally, terms that indicate the geometric shape of a component/surface encompass both exact and approximate shapes.

Although a target site is discussed herein as being in the subject's gastrointestinal tract, this disclosure is not so limited, as the target site may be any internal lumen, organ, cavity, or other tissue within the subject. Additionally, although endoscopes are referenced herein, it will be appreciated that the disclosure encompasses various devices that may be inserted into a body of a subject, such as ureteroscopes, cholangioscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, or catheters.

This disclosure is described with reference to exemplary medical devices, components, assemblies and/or systems having a handle and an insertion portion. It should be noted that reference to any device and/or any particular procedure is provided only for convenience and not intended to limit the disclosure. A person of ordinary skill in the art would recognize that the concepts underlying the disclosed devices and application methods may be utilized in any suitable procedure, medical or otherwise. The assemblies and systems described herein may be used in conjunction with other types of medical devices. This disclosure may be understood with reference to the following description and the appended drawings, wherein like elements are referred to with the same reference numerals.

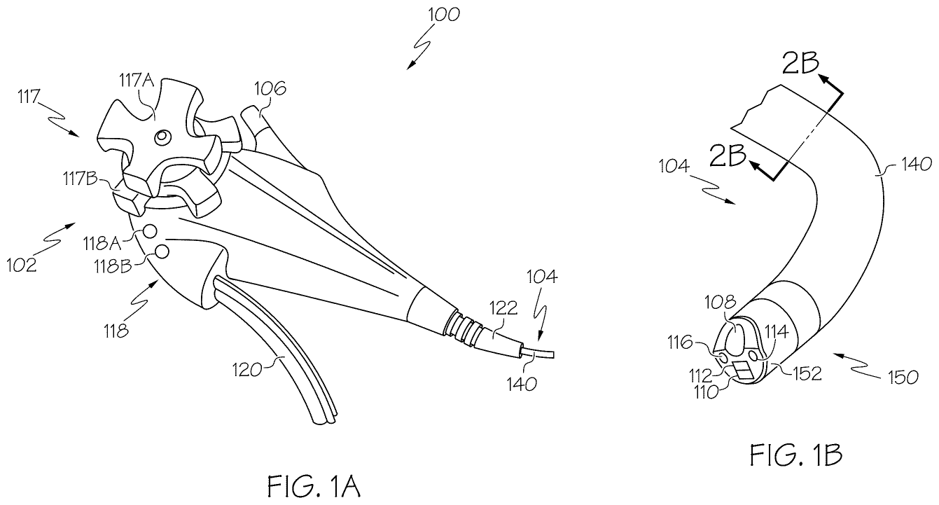

Referring to FIG. 1A, a medical device 100 according to an exemplary embodiment is shown. Medical device 100 includes a handle 102 and an insertion portion 104 (including, e.g., a shaft 140). Insertion portion 104 may be connected to and extend from a distal portion of handle 102. Insertion portion 104 may terminate distally in a distal end 150, as shown in FIG. 1B. In embodiments, the distal end of medical device 100 may include an end cap 152 that incorporates components at the distal end. Additionally, handle 102 includes an opening or a port 106 (e.g., a biopsy port), which may be fluidly connected to a working channel 108 and/or one or more lumens of insertion portion 104. As discussed in detail below, a portion of insertion portion 104 may be formed of shaft 140 (e.g., a multi-lumen extrusion), and only a portion of the shaft may extend proximally to port 106. For example, a first portion of shaft 140 may include a plurality of lumens, and a second portion of shaft may extend proximally to port 106 and may include only a subset of the lumens (e.g., one lumen).

Additionally, the distal end of medical device 100 (e.g., end cap 152) may include one or more illumination devices 110 (e.g., lights, LEDs, optical fibers, etc.) and/or one or more visualization devices 112 (e.g., cameras, imagers, etc.). Insertion portion 104 (e.g., shaft 140 and end cap 152) may also include one or more channels or lumens, for example, one or more fluid delivery lumens 114 and/or 116. Furthermore, as discussed in detail below, insertion portion 104 may include one or more additional channels or lumens, for example, to receive one or more pull or deflection wires, optical fibers, cables, electrical wires, communication wires, etc.

Handle 102 may include one or more actuators 117, for example, a first actuator 117A and a second actuator 117B. Actuators 117A, 117B may be manipulated (e.g., rotated about their respective axis) to control the deflection, movement, position, etc. of one or more portions of insertion portion 104, for example, distal end 150. In these aspects and as discussed in detail below, actuators 117A, 117B may be connected to one or more pull or deflection wires or cables that extend through one or more portions of insertion portion 104 and connect to distal end 150 of insertion portion 104.

Rotation of first actuator 117A and/or second actuator 117B may cause a portion of insertion portion 104 (e.g., distal end 150) to bend, for example, via an articulating joint (not shown). For example, rotation of first actuator 117A in a first direction (e.g., clockwise) may pull a first pull wire proximally, such that distal end 150 deflects in a first deflection direction (e.g., up). Rotation of first actuator 117A in a second direction (e.g., counterclockwise) may pull a second pull wire proximally, such that distal end 150 deflects in a second deflection direction (e.g., down). The second deflection direction may be opposite to the first deflection direction. Additionally, in some examples, rotation of second actuator 117B a first direction (e.g., clockwise) may pull a third pull wire proximally, such that distal end 150 deflects in a third deflection direction (e.g., left). Rotation of second actuator 117B in a second direction (e.g., counterclockwise) may pull a fourth pull wire proximally, such that distal end 150 deflects in a fourth deflection direction (e.g., right).

Handle 102 may also include one or more valves 118, for example, a first valve 118A and a second valve 118B. Although two valves (i.e., first valve 118A and second valve 118B) are shown, handle 102 may include additional (e.g., a third valve, a fourth valve, etc.) or fewer valves (e.g., no valves or only one of first valve 118A or second valve 118B). In some embodiments, first valve 118A may be configured to control the supply of air and/or water through insertion portion 104 to distal end 150. Second valve 118B may be configured to control the application of suction through insertion portion 104 to distal end 150 and to inject contrast and saline agents or solutions. Additional valves may be used, for example, to control the application of one or more medicines, agents, materials, etc. from distal end 150. In other aspects, one or more syringes or other fluid sources may be coupled to medical device 100, for example, via one or more ports (e.g., port 106 or other inlets) on handle 102.

An umbilicus 120 may extend from handle 102 (e.g., from a proximal portion of handle 102). Umbilicus 120 may be removably coupled (e.g., directly or indirectly) to a processing unit (not shown). The processing unit may be configured to process information (e.g., sensor data, imaging data, light data, etc.) received from medical device 100. In some aspects, the processing unit may be a controller associated with medical device 100. Illumination device(s) 110 and/or imaging device(s) 112 may be electrically coupled (e.g., directly or indirectly) to the processing unit, for example, via one or more wires and/or cables extending through insertion portion 104, through handle 102, and through umbilicus 120.

Handle 102 may be coupled to a proximal portion of insertion portion 104 (e.g., shaft 140) via a strain relief portion 122. Strain relief portion 122 may extend from a distal end of handle 102 and may overlap with a proximal portion of shaft 140, for example, helping to maintain the coupling of handle 102 to insertion portion 104.

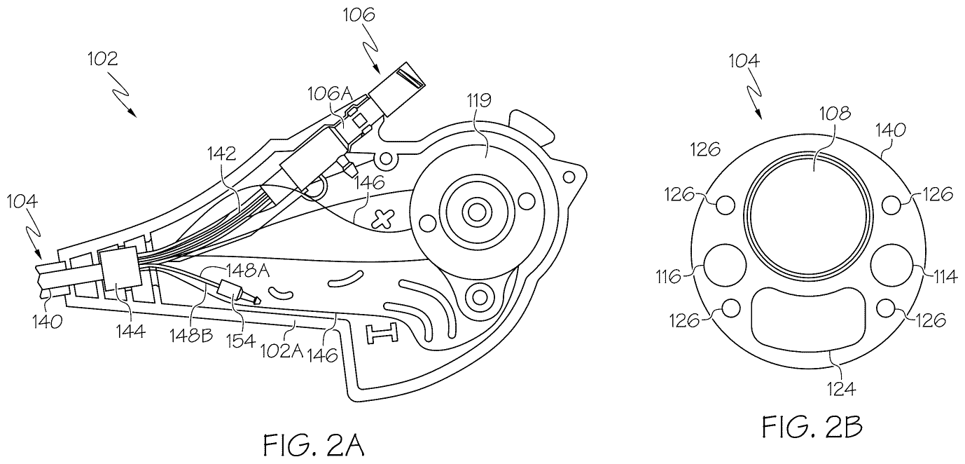

FIG. 2A is a side view of internal components of handle 102 and a portion of insertion portion 104, without umbilicus 120, and FIG. 2B is a lateral cross-sectional view of a portion of insertion portion 104. As shown in FIG. 2A, handle 102 includes a handle body 102A that encloses one or more components of medical device 100. For example, handle body 102A may be formed of two halves that are coupled together (e.g., adhered, welded, snap fit, etc.) to form handle body 102A. Moreover, handle 102 includes port 106, for example, including a port body 106A. A portion of shaft 140 may extend proximally to be positioned within a portion of handle body 102A. Furthermore, a portion of shaft 140 (e.g., a skived portion 142 or a portion that forms working channel 108, shown in FIG. 2B) extends through handle body 102A to be coupled to a portion of port 106 (e.g., to port body 106A). Skived portion 142 may include only a single complete lumen-working channel 108. In other words, working channel 108 may be an only complete lumen of skived portion 142.

Skived portion 142 may be separated from the remainder of shaft 140, for example, via cutting with a razor blade, a trimming tool, etc. The separation of skived portion 142 from the remainder of shaft 140 may be done, for example, under microscope or magnification. In these aspects, the additional lumens, or channels of shaft 140 may have exposed proximal ends to receive or be coupled to other components of medical device 100. For example, skived portion 142 may be formed by removing lumens 114 and/or 116, communication lumen 124, and one or more wire lumens 126 from a proximal portion of shaft 140.

As shown, medical device 100 includes one or more articulation, deflection, or pull wires 146, which may be coupled to one or more of actuators 117 (FIG. 1A), for example, to an internal actuator portion 119. Rotation of one or more of actuators 117 may also rotate internal actuator portion 119. As discussed above, rotation or manipulation of actuators 117 may thus also control one or more of pull wires 146 to control an articulation, movement, or position of a portion of insertion portion 104 (e.g., distal end 150). Although only one internal actuator portion 119 and two pull wires 146 are shown, it is noted that handle 102 may include two internal actuator portions (e.g., each coupled to one of actuators 117A and 117B), and that medical device 100 may include four pull wires 146 (e.g., with two pull wires 146 coupled to each internal actuator portion 119). Additionally, each pull wire 146 may extend through a lumen or channel within shaft 140 (as discussed below), for example, through respective proximal openings (e.g., adjacent to hub 144 and where skived portion 142 extends from the remainder of shaft 140).

Medical device 100 also includes one or more fluid tubes, for example, coupling portions of shaft 140 to one or more portions of handle 102. For example, medical device 100 may include a fluid tube 148, such as an irrigation tube, which may convey a fluid such as water or air to distal end 150. In some aspects, fluid tube 148 may be coupled (e.g., at a proximal ends) to an irrigation or air hub or adapter 154. Although not shown, medical device 100 may also include one or more additional fluid tubes for irrigation (water), air (insufflation), to inject contrast and saline agents or solutions, and/or for suction (aspiration). The one or more fluid tubes 148 may extend through or couple one or more fluid lumens in shaft 140 to one or more valves 118 (e.g., one tube extending to first valve 118A, and one tube extending to second valve 118B). In these aspects, valve(s) 118 may control the delivery of fluid and/or application of suction to a treatment site via one or more channels or lumens in shaft 140. In some aspects, adapter 154 may help to couple valve(s) 118 (FIG. 1A) to fluid tube 148 or other tubes.

As shown in FIG. 2B, insertion portion 104 includes shaft 140. As mentioned, shaft 140 may be a multi-lumen extrusion, for example, formed of polytetrafluoroethylene (PTFE) and/or additive-filled polymers, which may help to lubricate to one or more lumens (e.g., working channel 108). Shaft 140 may be formed of a single piece of material, e.g., to form a single, integral body. Lumens of shaft 140 may be formed integrally from shaft 140.

Shaft 140 includes working channel 108. Working channel 108 may be positioned in a central portion of shaft 140 or may be slightly off-center (as shown). Shaft 140 also may include one or more fluid delivery lumens 114 and/or 116. A communication lumen 124 may receive one or more cables, wires, etc. to electrically connect one or more of illumination device(s) 110 and/or imaging device(s) 112 to handle 102 and/or umbilicus 120. Shaft 140 also includes one or more wire lumens 126, for example, to movably receive respective pull wires 146 (FIG. 2A). Wire lumens 126 may be evenly or unevenly spaced around outer portions of shaft 140, for example, above or below one or more of lumens 114 and/or adjacent to (e.g., radially outside of) one or more of working channel 108 and/or communication lumen 124.

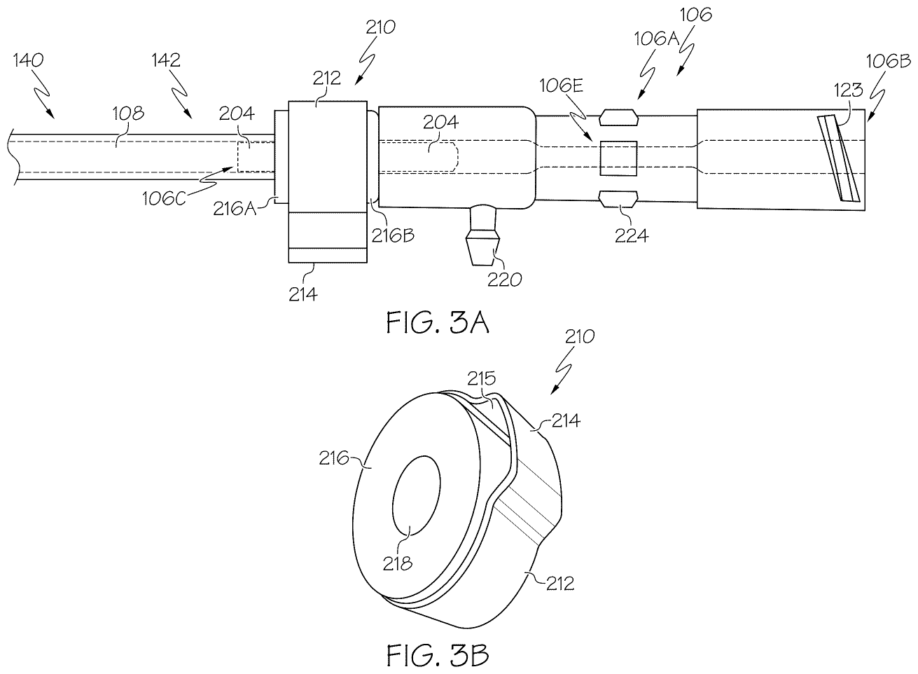

According to aspects of this disclosure, embodiments are described that may help prevent one or more leaks when coupling a portion of shaft 140 or skived portion 142 (e.g., working channel 108 or lumen) to a portion of the handle (e.g., to port 106). For example, the process of skiving or separating working channel 108 from other portions of shaft 140 described above may result in a non-concentric and/or non-circular outer diameter with respect to the working channel longitudinal axis. A leak may occur when there are variations in the size (e.g., the inner diameter (ID) or outer diameter (OD)) of port 106 and/or in the size (e.g., the inner diameter (ID) or outer diameter (OD)) of components of skived portion of shaft 140 (e.g., working channel 108), as the variations in size may lead to potential gaps or spaces where fluid can escape. In addition, irrigation and aspiration channels within handle 102 (i.e., formed by port 106 and working channel 108) may be subjected to pressures (e.g., up to approximately 40 psi) during operation, which may exacerbate gaps or spaces, causing fluid(s) to leak. Referring to FIG. 3A, a proximal portion of shaft 140, including skived portion 142, may be coupled to port 106 (e.g., on handle 102, FIGS. 1A and 2A) including a seal 210. Seal 210 may help to prevent leaks in the proximal portion of shaft 140 and port 106.

FIG. 3A illustrates a proximal portion of skived portion 142 of shaft 140 (including working channel 108, FIG. 2B) coupled to port 106 and separated from other components or portions of medical device 100, for example, handle 102. Port 106 may include a port body 106A. A proximal portion of port body 106A may include a projection 224 that may nest within a portion of handle 102 when port 106 is coupled to handle 102 (FIG. 2A).

The proximal portion of port body 106A may also include one or more surface features 123, for example, threading or grooves, which may help a user couple a portion of an instrument to port body 106A. Surface features 123 may help to couple or accept the internal grooves of an instrument, such as a syringe that injects fluid into port 106. The fluid may act as a source of irrigation and flow distally into working channel 108 of shaft 140. Port 106 may also include a suction port 220 that may connect to a tube or a hose external to handle 102 (not shown) that may provide for an application of suction by the medical device 100. Alternatively or additionally, surface features 123 may help to couple or accept internal grooves of a medical instrument, for example, a grasper, a basket, an electrosurgical knife, etc.

Port body 106A includes a proximal opening 106B and a distal opening 106C. Port body 106A also includes a port lumen 106E, for example, extending the length of and fluidly connecting proximal opening 106B to distal opening 106C. Medical instruments (e.g., an accessory device) may be delivered through port body 106A (e.g., proximal opening 106B and port lumen 106E to distal opening 106C). When port 106 and skived portion 142 of shaft 140 are coupled, medical instruments may be introduced into working channel 108 of shaft 140 and extend out of the distal opening of working channel 108. The proximal-most end of skived portion 142 and working channel 108 of shaft 140 may be distal of port body 108A; for example, the proximal-most end of skived portion 142 and working channel 108 may be surrounded by seal 210, described below.

Prior to coupling skived portion 142 of shaft 140 (including working channel 108) to port 106, a hypotube 204 may be introduced through distal opening 106C of port body 106A. Hypotube 204 may be generally cylindrical, or one or more shapes, for example, aligning with or at least partially corresponding to the shape of working channel 108 and/or port body 106A. A proximal portion of hypotube 204 may be positioned within a distal portion of port body 106A but still distal to suction port 220. Port lumen 106E and hypotube 204 may be sized and/or shaped such that at least a portion of hypotube 204 received within port lumen 106E may form a press fit coupling. For example, hypotube 204 may be molded to conform to the shape of port lumen 106E. Hypotube 204 may also be overloaded into place with excess force when inserting hypotube 204 into port lumen 106E to help promote a tight and secure fit within port lumen 106E.

Furthermore, adhesive (e.g., one or more adhesive layers) may be applied, delivered, or positioned around one or more of port lumen 106E and hypotube 204. The adhesive may be applied on a portion of an outer surface (e.g., a radially outer surface) of hypotube 204 to help secure hypotube 204 within port body 106A. For example, the adhesive may be applied on a wall (e.g., an inner surface of the wall) of port lumen 106E. Furthermore, one or more portions of hypotube 204 may include surface features to allow for increased surface roughness and/or promote a seal with the one or more layers of adhesive, thus helping to minimize the chances of a leak. For example, one or more portions of hypotube 204 may be treated by one or more of: sanding, laser texturing, film wrapping, wire wrapping, including one or more stops (e.g., in port 106 and/or hub 144), etc.

Subsequently, hypotube 204 may be positioned within the portion of shaft 140 (e.g., skived portion 142) that forms working channel 108. Working channel 108 may radially surround hypotube 204. Once hypotube 204 is introduced into working channel 108, a portion of hypotube 204 extends distal of port body 106A and may extend distal to seal 210. However, the disclosure is not so limited, and hypotube 204 may vary in length based on the requirements of a medical procedure. For example, hypotube 204 may be longer or extend further distally through working channel 108 than as shown in FIG. 3A. A longer hypotube 204 may help working channel 108 of shaft 140 maintain its shape and/or orientation as shaft 140 is guided through the body's internal passages. However, a longer hypotube 204 may make the shaft 140 less flexible. In any embodiment, working channel 108 may radially surround a portion of hypotube 204 that extends distally of port body 106A.

As seen in FIG. 3A, skived portion 142 of shaft 140 (including working channel 108) may be coupled to port 106, for example, at least partially via seal 210. FIG. 3B shows seal 210 uncoupled from port 106 and shaft 140. Seal 210 may include a coupling assembly including at least two pieces. For example, the coupling assembly of seal 210 may include an outer clamp 212 and at least one internal ring 216. Outer clamp 212 and internal ring 216 may be two discrete pieces or components.

Outer clamp 212 may have a generally cylindrical shape and/or a shape compatible with an internal ring 216, for example, a shape that helps outer clamp 212 encircle ring 216, as described below. Outer clamp 212 may optionally include a protrusion 214. In an embodiment, outer clamp 212 may optionally include one or more protrusions 214 projecting outwardly of outer clamp 212. Protrusion 214 may also include one or more openings 215, forming an inner surface of protrusion 214, and defining a semi-rectangular shape of protrusion 214. As shown in FIGS. 3A and 3B, in some aspects, outer clamp 212 may include one protrusion 214 with a corresponding one opening 215. Nevertheless, in other aspects, outer clamp 212 may include more than one protrusions 214. In some embodiments more than one outer clamp 212 may be used. Outer clamp 212 may be composed of a rigid material such as stainless steel, plastic, or any other suitable material that enables outer clamp 212 to help secure seal 210 and/or compress at least one internal ring 216 positioned within outer clamp 212.

Alternative assemblies and/or methods for constricting and securing internal ring 216 (e.g., to help form seal 210) include using one or more zip ties, one or more Tuohy Borst style caps, one or more spring band clips, one or more worm drive hose clamps, and/or one or more suitable structures. In these aspects, seal 210 may help to adjustably compress and/or securely hold internal ring 216 to couple skived portion 142 of shaft 140 and port 106.

Internal ring 216 is at least partially compliant and includes an opening 218 designed (e.g., having a suitable size and shape) to encircle the portion of shaft 140 that forms working channel 108. As mentioned, shaft 140 may be a multi-lumen extrusion, for example, formed of polytetrafluoroethylene (PTFE) and/or additive-filled polymers. Internal ring 216 may be composed of molded liquid silicone rubber or thermoplastic elastomer (TPE). Internal ring 216 may be a circular structure consisting of a silicone tube, which may comprise molded liquid silicone rubber or thermoplastic elastomer. For example, internal ring 216 may be an O-ring, a tubing element, or an elastic material with a hollow lumen.

In these aspects, the material(s) of internal ring 216 may be compatible with the material(s) of shaft 140. For example, the material(s) of internal ring 216 may help to engage with or otherwise interact or manage the material properties of the material(s) (e.g., PTFE) that form shaft 140. In some aspects, the material(s) of internal ring 216 may help to provide enhanced frictional properties or engagement with shaft 140, which may help to improve seal 210. The material(s) of internal ring 216 may also conform to irregular surfaces of skived portion 142. For example, outer clamp 212 may crimp or otherwise compress internal ring 216, which may help to maintain a tight seal around skived portion 142 of shaft 140 and port 106.

As mentioned above, outer clamp 212 and internal ring 216 may form seal 210. To form seal 210, opening 218 of internal ring 216 may be positioned at a desired location. Opening 218 of internal ring 216 may accommodate (e.g., have a suitable size and shape to be positioned around) the outer diameter of skived portion 142 of shaft 140 (including working channel 108). Specifically, an inner surface of internal ring 216 defined by opening 218 may be positioned on a radially outer surface of the proximal or proximal-most portion of skived portion 142 (including working channel 108) that is to be coupled to port 106. Outer clamp 212 may be placed on (i.e., around) internal ring 216 to clamp internal ring 216 at the desired location described above.

Subsequently, outer clamp 212 may be tightened or crimped to clamp or exert pressure on internal ring 216 and create seal 210 around skived portion 142 of shaft 140 (and a portion of hypotube 204), as shown in FIG. 3A. The compression of internal ring 216 against working channel 108 and skived portion 142 of shaft 140 (including working channel 108) helps to create a snug fit. As discussed, a leak may occur when there are variations in the size (e.g., the inner diameter (ID) or outer diameter (OD)) of port 106 and/or in the size (e.g., the inner diameter (ID) or outer diameter (OD)) of components of skived portion of shaft 140 (e.g., working channel 108), as the variations in size may lead to potential gaps or spaces where fluid can escape. Compression of internal ring 216 by outer clamp 212 may help secure seal 210, coupling shaft 140, and port 106 and prevent fluid from escaping from potential leak paths. For example, fluid, debris, or other material may escape between hypotube 204 and the inner surface of working channel 108 when suction occurs.

Seal 210 helps to fluidly and sealingly couple skived portion 142 of shaft 140 (including working channel 108) to port 106, thus helping to prevent fluid, debris, or other material from escaping. Additionally, applying adhesive around hypotube 204 and port lumen 106E, as described above, may help to enhance the sealing properties of the coupling. For example, the adhesive may form bonds or otherwise help to fill any remaining gaps and/or create an additional barrier against fluid leakage within the coupling between hypotube 204, port 106, and skived portion 142 of shaft 140. Additionally, the adhesive may help to stabilize hypotube 204, reducing the risk of movement or dislodgment.

In an embodiment, multiple internal rings 216 may be positioned on skived portion 142 of shaft 140 (including working channel 108), and multiple internal rings 216 may be secured by outer clamp 212. The internal rings 216 may be at a distal and a proximal end of outer clamp 212. For example, a distal internal ring 216A may be positioned onto both skived portion 142 of shaft 140 and the distal portion of hypotube 204, as shown in FIG. 3A. A proximal internal ring 216B may be proximal to distal internal ring 216A and may be positioned directly on hypotube 204. Proximal internal ring 216B may abut the distal end of port 106 to help seal distal opening 106C that receives hypotube 204. If a potential leak path exists between hypotube 204 and skived portion 142 of shaft 140, and the leak path is not fully sealed by the compression of distal internal ring 216A, the proximal internal ring 216B may help keep the leak contained.

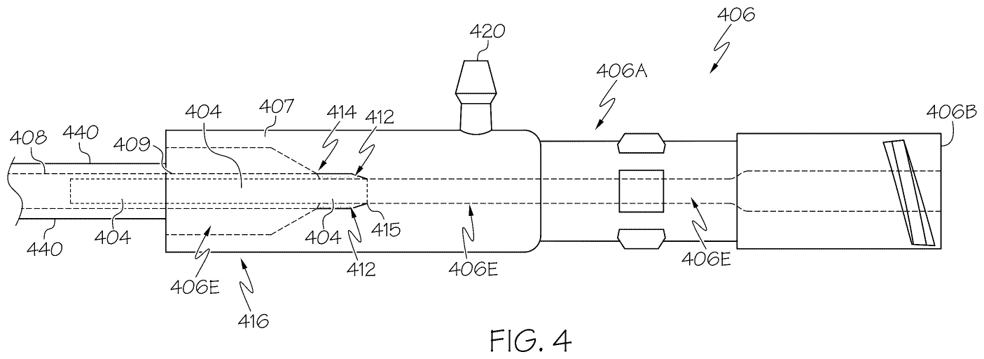

FIG. 4 illustrates another embodiment and coupling assembly used to help prevent a leak when coupling a working channel 408 of a shaft 440 (e.g., similar to working channel 108 of shaft 140) to a port 406. Port 406 may be similar to port 106 discussed above, except for the differences described herein.

A proximal portion of a port body 406A may be similar to port body 106A, including a suction port 420 (i.e., similar to suction port 220). Port body 406A includes a proximal opening 406B and a distal opening 406C. Port body 406A also includes a port lumen 406E extending the length of and fluidly connecting proximal opening 406B to distal opening 406C. Distal portion 407 and distal opening 406C may be wider than in previous embodiments (e.g., port body 106A) to form a volume (i.e., a space) to receive (e.g., contain) proximal portion of shaft 440, hypotube 404, and the one or more adhesives to help couple the components, as described below. Port lumen 406E may correspond to the dimensions of port body 406A. For example, port lumen 406E may taper at a midpoint of port body 406A and increase in width at distal portion 407 of port body 406A.

The portion of shaft 440 that forms working channel 408 may be similar to working channel 108 of shaft 140, except for the differences described herein. The portion of shaft 440 that forms working channel 408 may have a circular cross-sectional shape to be introduced into distal opening 406C of port body 406A. Port lumen 406E and portion of shaft 440 that forms working channel 408 may be sized and/or shaped such that at least the portion of shaft 440 that forms working channel 408 may be received within port lumen 406E. The portion of shaft 440 that forms working channel 408 may extend through port lumen 406E until it reaches an abutment 414. A proximal end face of the portion of shaft 440 including working channel 408 may abut one of the one or more abutments (e.g., abutment 414 and abutment 415). Abutment 414 may be a portion of port lumen 406E with a diameter less than the diameter an outer diameter of the proximal end of shaft 440. In other words, at least a portion of the proximal portion of the port lumen is narrower than at least a portion of the distal portion of the port lumen to form one or more abutments (e.g., abutment 414 and abutment 415).

Hypotube 404 may be similar to hypotube 204. Working channel 408 and hypotube 404 may be sized and/or shaped such that hypotube 404 may be received within a proximal portion of working channel 408 (i.e., hypotube 404 marked in dashes is within working channel 408). Hypotube 404 may extend proximally such that the proximal portion of hypotube 404 is within distal portion 407 of port body 406A but still distal to suction port 420. For example, the proximal portion of hypotube 404 may extend proximally past a proximal end of shaft 440 and abutment 414. In an embodiment, hypotube 404 may extend through port lumen 406E until it reaches an abutment 415 (i.e., similar to abutment 414). In some aspects, abutment 414 may act as a first narrowing portion of port lumen 406E that acts as a first stop surface for the proximal end of shaft 440. Subsequently, abutment 415 may act as a second narrowing portion of port lumen 406E that acts as a second stop surface for hypotube 404. Furthermore, as discussed below, hypotube 404 and a proximal portion of shaft 440 may be securely coupled to port 106 (e.g., within port lumen 106E) with one or more adhesives.

Adhesive (e.g., an adhesive layer) may be applied, delivered, or otherwise positioned around one or more of port body 406A, working channel 408, and hypotube 404. For example, adhesive may be applied on a portion of an outer surface 409 (e.g., a radially outer surface) of shaft 440 to help shaft 440 within port body 406A. In addition, adhesive may be applied on a wall 416 (e.g., an inner surface of wall 416) of port lumen 406E. The adhesive or adhesive layers may help secure the portion of shaft 440 that forms working channel 408 to distal portion 407 of port body 406A.

The portion of hypotube 404 extending proximally past working channel 408 and abutment 414 may include one or more layers 412 of adhesive between the outer surface of hypotube 404 and wall 416 (e.g., an inner surface of wall 416) of port lumen 406E. Accordingly, a distal portion of port lumen 406E may form a well to receive one or more adhesives to help couple respective portions of both hypotube 404 and shaft 440 within port body 406A. In some aspects, vacuum pressure may be used (e.g., applied to proximal opening 406B of port 406) to help draw the adhesive into or along port lumen 406E to help secure shaft 440 and hypotube 404 to port body 406A.

The portion of shaft 440 that forms working channel 408 may include one or more surface features to help the adhesives couple working channel 408 to port body 406A. For example, if shaft 440 (and thus working channel 408) is formed of PTFE, the proximal portion of shaft 440 may be etched (e.g., chemically etched), roughened, or otherwise textured to help the adhesive(s) couple shaft 440 to port body 406A. Alternatively, or additionally, one or more portions of hypotube 404 may include one or more surface features. For example, one or more portions of shaft 440 and/or hypotube 404 may be treated by one or more of: sanding, laser texturing, film wrapping, wire wrapping, including one or more stops (e.g., in port 106 and/or hub 144), etc.

Additionally, although not shown, during the coupling of the various components, port 406 may receive a mandrel. The mandrel may help to secure or otherwise hold or maintain an alignment of port body 406A and the portion of shaft 440 that forms working channel 408 during assembly. The mandrel may be sized and shaped to be received within port lumen 406E. For example, the mandrel may help to maintain the positioning of the various components during the delivery and/or curing of an adhesive, may help to prevent buckling or other changes in shape of hypotube 404 and/or the portion of shaft 440 that forms working channel 408 during heating (e.g., heat shrinking, heated drying or curing of the adhesive, etc.), or may otherwise help to retain the relative positions during assembly. In some aspects, skived portion 142 may be sealing coupled to distal portion 407 of port 406 with adhesive only, that is, without hypotube 404, for example, in situations where the inner diameter of the working channel 408 may vary in size or shape. Furthermore, the mandrel may help to prevent adhesive(s) from flowing into the inner lumen (e.g., working channel 408) of shaft 440.

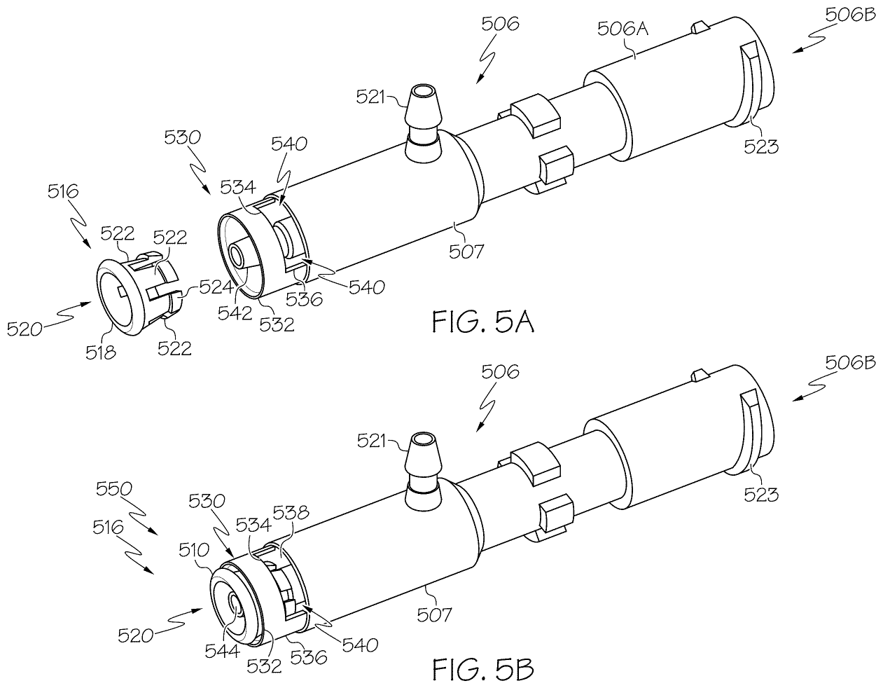

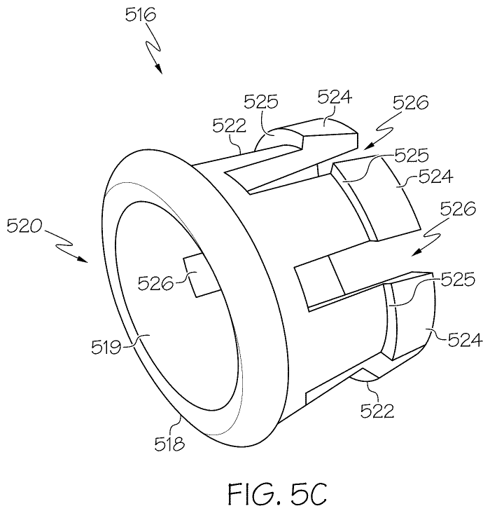

FIGS. 5A-5C illustrates components of a coupling assembly 550 that may be used to help couple a shaft with a working channel (e.g., shaft 140 with working channel 108, FIG. 2A-2B) to a port 506, including a port body 506A (e.g., similar to port body 106A), as discussed above. A proximal portion of port body 506A may be similar to port body 106A, including a suction port 521 (i.e., similar to suction port 220) and surface features 523 (i.e., similar to surface features 123), except for the differences described herein. Port body 506A includes a proximal opening 506B and a distal opening 506C (e.g., similar to proximal opening 206B and distal opening 206C).

FIG. 5A shows a connector 516 and port 506 of assembly 550 in a first uncoupled configuration. As shown, port 506 includes a ring 530. Connector 516 and ring 530 may be molded or machined. Ring 530 may be integral with or attachable to a distal portion 507 of port body 506A. For example, port body 506A and ring 530 may be a single piece. Alternatively, a proximal surface 538 of ring 530 may be friction fit within distal opening 506C of port body 506A. Ring 530 may include a distal ledge 532, a proximal ledge 534, a plurality of arms 536, a proximal surface 538, and a plurality of openings 540, as described below. Connector 516 and/or ring 530 may be formed of any rigid or semi-rigid material (e.g., one or more polymeric materials).

The longitudinal length of distal end of ring 530 may be defined from distal end of distal ledge 532 to the proximal end of proximal ledge 534. Arms 536 may extend proximally from a proximal edge of proximal ledge 534 to a distal face of proximal surface 538. Arms 536 may be adjacent and disposed or spaced apart at an interval relative to one another. The interval may define a plurality of openings 540 between proximal ledge 534 and proximal surface 538. The intervals of arms 536 may be regularly or irregularly spaced to form opening(s) 540. The size and/or shape of the interval(s) defining a plurality of openings 540 and other aspects of ring 530 (e.g., the width of the distal end of ring 530) may be based on the dimensions or features of connector 516, described below.

Ring 530 may also include a projection 542. Projection 542 may extend from proximal surface 538 to a distal end of ring 530, for example, radially within at least a portion of one or more of distal ledge 532 and proximal ledge 534. Projection 542 may taper from a proximal end to a distal end. Projection 542 may include an opening 544, for example, extending through a length of projection 542. In some aspects, the tapering of projection 542 may help to couple a lumen of working channel 108 on port body 506A. The distalmost end of projection 542 may have a diameter smaller (i.e., increasing diameter more proximally) than the lumen of working channel 108, enabling a snug fit.

FIGS. 5A-5C also illustrate connector 516, and FIG. 5C depicts connector 516 separate from ring 530 and the rest of port 506. As shown in FIG. 5C, connector 516 may include a distal ring 518, a through-hole 520, and a plurality of fingers 522. The size of through-hole 520 of distal ring 518 may be selected based on the outer diameter of, for example, working channel 108 described above. Fingers 522 extend proximally from distal ring 518 and may be disposed or spaced apart at a plurality of intervals 526 relative to one another. Fingers 522 may include a raised ledge 524, including a stop surface 525. Raised ledge 524 may be a size and shape that allows it to receive at least a portion of proximal ledge 534 of ring 530. Fingers 522 may be cantilevered projections. For example, fingers 522 may extend proximally from a proximal end of distal ring 518, and stop surfaces 525 may be formed by radial extensions at the proximal portions of each of fingers 522.

FIG. 5B shows assembly 550, with connector 516 coupled to ring 530 of port 506. Connector 516 may be coupled to ring 530 by introducing fingers 522 into a plurality of openings 540 of ring 530. Fingers 522 may move radially inward along proximal ledge 534 of ring 530 until proximal ledge 534 reaches stop surface 525 of fingers 522 (FIG. 5C). Once proximal ledge 543 reaches stop surface 525, raised ledge 524 may extend radially outward, snap onto, or otherwise engage with the surface of proximal ledge 534 of ring 530. In other words, connector 516 may be configured to form a snap-fit coupling to ring 530. At this position, stop surface 525 may abut with proximal ledge 534, such that connector 516 is interlocked and/or fixedly coupled to ring 530 to form assembly 550. Additionally, as shown in FIG. 5B, once connector 516 is coupled to ring 530, opening 544 of connector 516 at least partially overlaps with projection 542. Once assembly 550 is formed, a portion of shaft 140 (e.g., a skived portion 142 or a portion that forms working channel 108, shown in FIG. 2B) may be coupled to port 506.

For example, the proximal portion of shaft 140 (e.g., a skived portion 142 or a portion that forms working channel 108, shown in FIG. 2B) may be introduced and positioned around the distal end of projection 542 such that through-hole 520 aligns with working channel 108 of the proximal portion of shaft 140. Working channel 108 of shaft 140 may then radially surround projection 542. Inner surface 519 of distal ring 518 may help to provide a friction fit while coupled with the outer diameter of the portion of shaft 140. Subsequently, connector 516 may be placed onto the proximal portion of shaft 140 such that opening 544 passes over shaft 140. As discussed, fingers 522 of connector 516 may then move radially inward along proximal ledge 534 of ring 530 to interlock with ring 530 and radially surround the distal end of projection 542. In these aspects, shaft 140 may be fixedly placed between projection 542 and opening 544 of projection 542. Opening 544 may be fluidly connected to working channel 108 of shaft 140.

Assembly 550 may help to form a seal, for example, between working channel 108 of the proximal portion of shaft 140 and port 506. Medical instruments (e.g., an accessory device) may be delivered through port body 506A (e.g., proximal opening 506B to distal opening 506C). When port 506, assembly 550, and working channel 108 of shaft 140 are coupled, medical instruments may be introduced through opening 506B of port 506 (i.e., via a port lumen not shown), and then through opening 544 of projection 542. Subsequently, medical instruments may extend through working channel 108 of shaft 140 until the medical instrument extends out of the distal end of working channel 108 (e.g., shown in FIG. 1B).

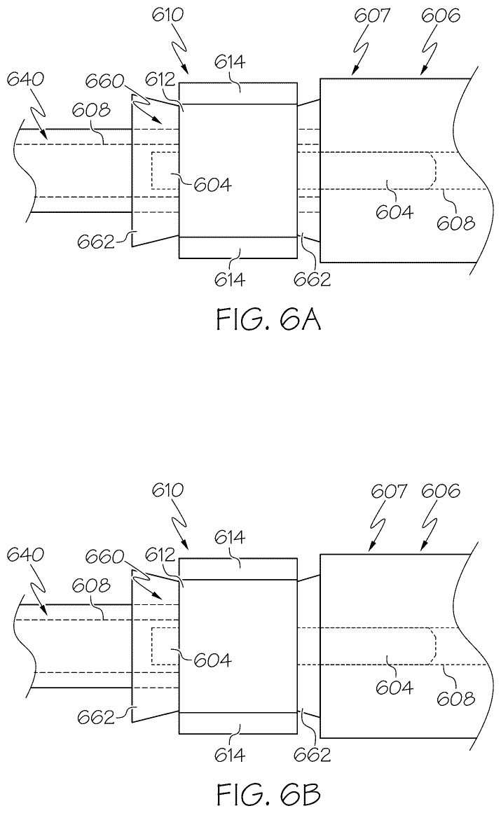

FIGS. 6A and 6B illustrate enlarged views of another coupling assembly 610 used to help prevent a leak when coupling a working channel 608 of a shaft 640 (e.g., similar to working channel 108 of shaft 140, except for the differences described herein) to a port 606. Port 606 may be similar to port 406, except for the differences described herein. For illustrative purposes, only a distal portion 607 of port 606 is shown. However, a proximal portion of port 606 may be similar to the proximal portion of port 406, for example, including a suction port.

In FIGS. 6A and 6B, distal portion 607 of port 606 may include a hypotube 604 radially surrounded by working channel 608. Hypotube 604 may be generally cylindrical, or one or more shapes, for example, aligning with or at least partially corresponding to the shape of distal portion 607. In some aspects distal portion 607 of port 606 may be overmolded or otherwise formed on hypotube 604. For example, a thermoplastic or elastomeric material, may be injected or molded around hypotube 604 to form a connection between port 606 and the hypotube 604. Similar to hypotube 404, hypotube 604 may also extend proximally or be overmolded such that the proximal portion of hypotube 604 is within distal portion 607 but still distal to a suction port, as discussed above.

Working channel 608 and hypotube 604 may be sized and/or shaped such that hypotube 604 may be received within a proximal portion of working channel 608 (i.e., hypotube 604 (shown in dashed lines) is within working channel 608 and port 606). Working channel 608 may radially surround hypotube 604. Similar to hypotube 204, Hypotube 604 may extend distal of distal portion 607 of port 606 and extend distal of coupling assembly 610. As shown in FIG. 6A, shaft 640 may be coupled to distal portion 607 of port 606, including hypotube 604, at least partially via coupling assembly 610. As described above regarding shaft 440, shaft 640 may include one or more surface features to help couple to port 606, for example, via hypotube 604. For example, a proximal portion of shaft 640 may be positioned radially around a portion of hypotube 604 that extends distally of port 606.

In an embodiment, coupling assembly 610 may form a seal and include at least two pieces to form the seal. For example, coupling assembly 610 may include an outer tube 660 and an outer crimping tube 612. Outer tube 660 and crimping tube 612 may be two discrete pieces or components.

Outer tube 660 may be a transparent polyurethane tubing or a suitable flexible material. Outer tube 660 may have an opening and be introduced such that it may radially surround at least a portion of shaft 640. In these aspects, the material(s) of outer tube 660 may be compatible with the material(s) of shaft 640. For example, the material(s) of outer tube 660 may help to engage with or otherwise interact or manage the material properties of the material(s) (e.g., PTFE) that form shaft 640. In some aspects, the material(s) of outer tube 660 may help to provide enhanced frictional properties or engagement with shaft 640.

Outer crimping tube 612 may have a generally cylindrical shape and/or be sized to have a shape compatible with outer tube 660. Outer crimping tube 612 may optionally include a plurality of extensions 614. For example, there may be two extensions 614 positioned opposite to one another on the outer crimping tube. Outer crimping tube 612 may be composed of a rigid material such as stainless steel, plastic, or any other suitable material that enables outer crimping tube 612 to help secure and/or compress outer tube 660. For example, as discussed below, outer crimping tube 612 may be crimped or otherwise compressed to help sealingly couple shaft 640 to port 606, and the crimping or compression may form one or more (e.g., two) extensions 614.

In an embodiment shown in FIG. 6B, the features of FIG. 6A may be similar, except as described herein. As shown in FIG. 6B, the proximal end of the working channel 608 may be within the boundaries of (e.g., between the proximal and distal ends of) the outer crimping tube 612. In FIG. 6B, distal portion 607 of port 606 does not include a hypotube 604 radially surrounded by working channel 608. In other words, outer crimping tube 612 may help to provide a seal for working channel 608 that does not extend to distal portion 607.

As mentioned above, outer tube 660 and outer crimping tube 612 may form coupling assembly 610. In these aspects, outer crimping tube 612 may help to adjustably compress and/or securely hold outer tube 660 to couple the skived portion of shaft 640, including working channel 608 to port 606. To help form the seal, the outer tube 660 may be positioned at a desired location of shaft 640. Specifically, an inner surface of outer tube 660 may be positioned or introduced to be on a radially outer surface of the proximal portion of shaft 640 (including working channel 608) to be coupled to port 606. Outer crimping tube 612 may be placed on the proximal portion of the outer tube 660. Outer crimping tube 612 may be placed on the outer tube 660 (which surrounds shaft 640) such that the proximal end of working channel 608 are within the boundaries of outer crimping tube 612. Alternatively, outer crimping tube 612 may be positioned anywhere along the length of outer tube 660 that may be suitable to help seal or prevent a potential leak path.

Subsequently, outer crimping tube 612 may be tightened or crimped to clamp or exert pressure on outer tube 660 and create the seal around shaft 640 (and a portion of hypotube 604), as shown in FIGS. 6A and 6B. In an embodiment, a die-set and/or a pneumatic crimper may be used to compress outer crimping tube 612 around outer tube 660. Compression of outer crimping tube 612 may help to both size the crimping tube 612 to align with the size and/or shape of the outer tube 660 and to the size and/or shape of the shaft 640. Compression of outer tube 660 may form flares 662 that expand outwardly due to the flexibility of the outer tube 660. In an embodiment, there may be two flares 662. The flare 662 on the proximal side may optionally abut port 606. Once assembly is complete, coupling assembly 610 may help to form a seal between the shaft 640 and the port 606 to fluidly connect working channel 608 to port 606.

Additionally, although not shown, during the coupling of the various components, port 606 may receive a mandrel. As described above, the mandrel may help to secure or otherwise hold or maintain an alignment of port 606 and the portion of shaft 640 that forms working channel 608 during assembly. The mandrel may be sized and shaped to be received within port 606. For example, the mandrel may help to in the assembly of various positions of positioning of hypotube 604 and/or the portion of shaft 640 that forms working channel 608 to retain the relative positions during assembly (i.e., position of working channel 60. In some aspects, shaft 640 may be sealing coupled to distal portion 607 of port 606 with adhesive only, that is, without hypotube 604, for example, in situations where the inner diameter of the working channel 608 may vary in size or shape.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A medical device, comprising:
a handle, wherein the handle includes a port, and wherein the port includes a port lumen;
a shaft extending from a portion of the handle, wherein a first portion of the shaft includes a plurality of first lumens and a second lumen, wherein a second portion of the shaft includes only the second lumen, and wherein the second portion of the shaft is proximal of the first portion of the shaft; and
a coupling assembly that couples a proximal end of the second portion of the shaft to the port, wherein the coupling assembly forms a seal between the proximal end of the shaft and the port to fluidly connect the port lumen to the second lumen.

2. The medical device of claim 1, wherein the coupling assembly includes a ring and a connector, wherein the ring includes a proximal surface coupled to a distal portion of the port, and wherein the connector is configured to form a snap fit coupling to the ring to couple the proximal end of the second portion of the shaft to the port.

3. The medical device of claim 2, wherein the ring includes a distal ledge, a proximal ledge, and a plurality of arms, wherein each of the plurality of arms extend proximally from the proximal ledge to the proximal surface of the ring.

4. The medical device of claim 3, wherein the ring includes a projection that extends distally from the proximal surface of the ring radially within at least a portion of the distal ledge and the proximal ledge.

5. The medical device of claim 3, wherein adjacent arms of the plurality of arms are disposed at an interval defining a plurality of openings between the proximal ledge and the proximal surface.

6. The medical device of claim 2, wherein the connector includes a connector ring, a through-hole, and a plurality of fingers.

7. The medical device of claim 6, wherein the plurality of fingers extends proximally from the connector ring, and wherein adjacent fingers are spaced apart by an interval.

8. The medical device of claim 6, wherein each finger of the plurality of fingers includes a raised ledge and a stop surface in a proximal portion of the respective finger.

9. The medical device of claim 1, wherein the coupling assembly includes at least one internal ring, an outer clamp, and a hypotube.

10. The medical device of claim 9, wherein the at least one internal ring includes an opening positioned on a radially outer surface of a portion of the second portion of the shaft.

11. The medical device of claim 10, wherein the outer clamp is configured to compress the at least one internal ring to couple the second portion of the shaft to the port, and wherein a proximal end of the at least one internal ring abuts a distal end of the port.

12. The medical device of claim 9, wherein the at least one internal ring includes a plurality of internal rings, wherein the plurality of internal rings is positioned on a radially outer surface of a portion of the second portion of the shaft, wherein the outer clamp compresses the plurality of internal rings, and wherein one of the plurality of internal rings is a proximal internal ring abutting a distal end of the port.

13. The medical device of claim 1, wherein the coupling assembly includes a hypotube, wherein a proximal portion of the hypotube is positioned within a distal portion of the port, wherein a distal portion of the hypotube is positioned within a proximal portion of the second lumen of the shaft.

14. The medical device of claim 13, wherein the coupling assembly further includes an adhesive, wherein the adhesive is positioned at least between a portion of an outer surface of the hypotube and a portion of an inner surface of the port lumen.

15. The medical device of claim 13, wherein the port lumen includes a distal portion and a proximal portion, wherein at least a portion of the proximal portion of the port lumen is narrower than at least a portion of the distal portion of the port lumen to form one or more abutments, wherein a proximal portion of the second portion of the shaft extends through a portion of the port lumen, and wherein a proximal end face of the second portion of the shaft abuts one of the one or more abutments, and
wherein the port includes a suction port extending from the port lumen, and wherein a proximal end of the hypotube is distal to the suction port.

16. A medical device, comprising:
a handle, wherein the handle includes a port, and wherein the port includes a port lumen;
a coupling assembly including a ring and a connector;
wherein the ring includes a distal ledge, a proximal ledge, a plurality of arms, and a proximal surface, and wherein the proximal surface of the ring is coupled to a distal portion of the port;
wherein the connector includes a connector ring, and a plurality of fingers each including a raised ledge and a plurality of stop surfaces; and
wherein, when the connector is coupled to the ring, the plurality of fingers is positioned radially inward along the proximal ledge such that the proximal ledge reaches stop surfaces of each of the plurality of fingers and the raised ledge engages with a surface of the proximal ledge of the ring to form the coupling assembly.

17. The medical device of claim 16, wherein the ring includes a projection that extends from the proximal surface of the ring radially within at least a portion of the distal ledge and the proximal ledge, and
wherein the connector ring includes an opening that at least partially overlaps with the projection when the connector is coupled to the ring.

18. The medical device of claim 17, wherein the projection tapers from a proximal end to a distal end, wherein the projection includes an opening extending through a length of the projection, and wherein the opening of the projection is fluidly connected to the port lumen.

19. The medical device of claim 18, further comprising:
a shaft, wherein the shaft extends from a portion of the handle, wherein a first portion of the shaft includes a plurality of first lumens and a second lumen, wherein a second portion of the shaft only includes the second lumen, wherein a proximal portion of the second portion of the shaft is positioned around the distal end of the projection, and wherein the coupling assembly forms a seal between the proximal end of the shaft and the port.

20. A medical device, comprising:
a handle, wherein the handle includes a port, and wherein the port includes a port lumen;
a shaft extending from a portion of the handle, wherein a first portion of the shaft includes a plurality of first lumens and a second lumen, and wherein a second portion of the shaft only includes the second lumen;
a hypotube, wherein a proximal portion of the hypotube is positioned within a distal portion of the port, wherein a distal portion of the hypotube is positioned within a proximal portion of the second lumen of the shaft;
a coupling assembly including at least one outer crimping tube and an outer tube;
wherein the outer tube is positioned on a radially outer surface of a portion of the second portion of the shaft; and
wherein the outer crimping tube is positioned on the outer tube and the outer crimping tube and the outer tube form a seal between the second portion of the shaft and the port to fluidly connect the second lumen to the port lumen.
